(19) United States
(12) Patent Application Publication
(10) Pub. No.: US 2002/0058034 A1
(43) Pub. Date: May 16, 2002

(54) Inhibition of differentiation of cytotoxic T-cells by P-selectin ligand (PSGL) antagonists

(75) Inventors: Narasimhaswamy Manjunath, Roslindale, MA (US); Ulrich Hans von Andrian, Brookline, MA (US)

Correspondence Address: LAHIVE & COCKFIELD, LLP, 28 State Street, Boston, MA, 02109, US

(73) Assignee: Genetics Institute, Inc.

(21) Appl. No.: 09/904,710

(22) Filed: Jul. 12, 2001

Related U.S. Application Data
(62) Division of application No. 09/431,979, filed on Oct. 29, 1999.
(60) Provisional application No. 60/106,315, filed on Oct. 30, 1998.

(51) Int. Cl. A61K 39/395; A61K 38/17; A61K 31/726

(52) U.S. Cl. 424/144.1; 514/12; 514/54

(57) ABSTRACT

Methods are disclosed for inhibiting the differentiation of an activated T-cell into a cytotoxic lymphocyte in a mammalian subject, comprising administering to a subject a therapeutically effective amount of a PSGL antagonist.

### BACKGROUND OF THE INVENTION

P-selectin is a cell adhesion molecule expressed, among other places, on vascular endothelium. Interaction of P-selectin with its ligand, PSGL (also known as “PSGL-1”, which is expressed, among other places, on neutrophils), causes cells circulating in the vasculature which express PSGL to attach to the endothelium, where other adhesion molecules mediate extravasation into the surrounding tissues. Thus, the P-selectin/PSGL interaction has been a well-documented step in the development of inflammatory and immune responses.

PSGL has been cloned and well-characterized as described in International Application No. WO98/08949 (which is incorporated herein by reference). Such application discloses polynucleotides encoding various forms of PSGL, including numerous functional soluble forms of PSGL. Thus, PSGL is a well-characterized molecule, the soluble forms of which are particularly amenable to administration as therapeutics.

Therefore, it would be desirable to determine whether PSGL is involved in other cellular interactions for which forms of PSGL or other PSGL antagonists could serve as inhibitors.

### SUMMARY OF THE INVENTION

Applicants have for the first time determined that soluble PSGL or antibodies directed to PSGL will inhibit the differentiation of activated proliferating T-cells into cytotoxic lymphocytes. Thus, soluble PSGL, PSGL antibodies and other PSGL antagonists will inhibit such differentiation and the attendant immune and inflammatory responses resulting therefrom. As a result, these antagonists can be used to treat diseases and other conditions which result from undesirable or over-aggressive immune and inflammatory responses, such as, for example, in allergic reactions and autoimmune conditions.

The present invention provides a method of inhibiting the differentiation of an activated T-cell into a cytotoxic lymphocyte in a mammalian subject, said method comprising administering to said subject a therapeutically effective amount of a PSGL antagonist.

Other embodiments provide for a method of treating or ameliorating an autoimmune condition, said method comprising administering to said subject a therapeutically effective amount of a PSGL antagonist.

Yet other embodiments provide for a method of treating or ameliorating an allergic reaction, said method comprising administering to said subject a therapeutically effective amount of a PSGL antagonist.

Other embodiments provide a method of treating or ameliorating asthma, said method comprising administering to said subject a therapeutically effective amount of a PSGL antagonist.

In each of such methods, said PSGL antagonist is preferably selected from the group consisting of a soluble form of PSGL, an antibody directed to PSGL, an antibody directed to sLeₓ, an antibody directed to sulfated tyrosine, sLeₓ, mimetics which inhibit sLeₓ binding and a small molecule inhibitor of PSGL binding. Soluble forms of PSGL and antibodies directed to PSGL are most preferred. Among soluble forms of PSGL, those preferred are soluble forms of PSGL comprising the first 19 amino acids of the mature amino acid sequence of PSGL, with forms comprising the first 47 amino acids of the mature amino acid sequence of PSGL being more preferred. In certain other preferred embodiments, such 47 amino acids are fused to the Ig portion of an immunoglobulin chain.

### DETAILED DESCRIPTION OF PREFERRED EMBODIMENTS

All patent and literature references cited are incorporated herein by reference as if fully set forth.

Numerous soluble forms of PSGL, including fusion proteins comprising PSGL sequence, are disclosed in International Application No. WO98/08949. Soluble forms of PSGL can be made in accordance with the methods disclosed therein and other methods known to those skilled in the art.

As used herein, the term “antibody” includes a polyclonal antibody, a monoclonal antibody, a chimeric antibody, a single-chain antibody, a CDR-grafted antibody, a humanized antibody or fragments thereof which bind to the indicated protein. Such term also includes any other species derived from an antibody or antibody sequence which is capable of binding the indicated protein.

Antibodies to a particular protein can be produced by methods well known to those skilled in the art. For example, monoclonal antibodies can be produced by generation of antibody-producing hybridomas in accordance with known methods (see for example, Goding. 1983. Monoclonal antibodies: principles and practice. Academic Press Inc., New York; Yokoyama. 1992. “Production of Monoclonal Antibodies” in Current Protocols in Immunology. Unit 2.5. Greene Publishing Assoc. and John Wiley & Sons). Polyclonal sera and antibodies can be produced by inoculation of a mammalian subject with the relevant protein or fragments thereof in accordance with known methods. Fragments of antibodies, receptors or other reactive peptides can be produced from the corresponding antibodies by cleavage of and collection of the desired fragments in accordance with known methods (see for example, Goding, supra; Andrew et al. 1992. “Fragmentation of Immunoglobulins” in Current Protocols in Immunology. Unit 2.8. Greene Publishing Assoc. and John Wiley & Sons). Chimeric antibodies and single chain antibodies can also be produced in accordance with known recombinant methods (see for example, U.S. Pat. Nos. 5,169,939, 5,194,594 and 5,576,184). Humanized antibodies can also be made from corresponding murine antibodies in accordance with well known methods (see for example, U.S. Pat. Nos. 5,530,101, 5,585,089 and 5,693,762).

“sLeₓ” is sialyl Lewis x, a carbohydrate involved in PSGL binding (see, WO98/08949). Methods of making sLeₓ are known to those skilled in the art. “Mimetics which inhibit sLeₓ binding” include carbohydrate and peptido/carbohydrate species which bind to determinants which bind sLeₓ in such a manner to inhibit sLeₓ binding (see, for example, U.S. Pat. No. 5,614,615). Other methods for making such mimetics are known in the art. The ability of such species to perform in the methods of the present invention can be determined by testing such species in the models described herein for testing of soluble PSGL and PSGL antibodies.

Small molecules which inhibit PSGL binding can also be identified by testing of candidate materials in the models described herein. Numerous compounds are available for testing to determine which perform in accordance with the present invention.

Pharmaceutical compositions containing a PSGL antagonist which are useful in practicing the methods of the present invention may also contain pharmaceutically acceptable carriers, diluents, fillers, salts, buffers, stabilizers and/or other materials well-known in the art. The term “pharmaceutically acceptable” means a material that does not interfere with the effectiveness of the biological activity of the active ingredient(s) and that is not toxic to the host to which it is administered. The characteristics of the carrier or other material will depend on the route of administration.

It is currently contemplated that the various pharmaceutical compositions should contain about 0.1 micrograms to about 1 milligram per milliliter of the active ingredient.

Administration can be carried out in a variety of conventional ways. Intraperitoneal injection is the preferred method of administration. Intravenous, cutaneous or sub-cutaneous injection may also be employed. For injection, the PSGL antagonist will preferably be administered in the form of pyrogen-free, parenterally acceptable aqueous solutions. The preparation of such parenterally acceptable protein solutions, having due regard to pH, isotonicity, stability and the like, is within the skill of the art.

The amount of PSGL antagonist used for treatment will depend upon the severity of the condition, the route of administration, the reactivity of the antagonist or the activity of the antagonist, and ultimately will be decided by the treatment provider. In practicing the methods of treatment of this invention, a therapeutically effective amount of a PSGL antagonist is administered. The term “therapeutically effective amount” means the total amount of each active component of the method or composition that is sufficient to show a meaningful patient benefit (e.g., curing, ameliorating, inhibiting, delaying or preventing onset of, preventing recurrence or relapse of). One common technique to determine a therapeutically effective amount for a given patient is to administer escalating doses periodically until a meaningful patient benefit is observed by the treatment provider. When applied to an individual active ingredient, administered alone, the term refers to that ingredient alone. When applied to a combination, the term refers to combined amounts of the active ingredients that result in the therapeutic effect, whether administered in combination, serially or simultaneously. A therapeutically effective dose of a PSGL antagonist in this invention is contemplated to be in the range of about 0.05 mg/kg to about 25 mg/kg, preferably about 1 mg/kg to about 20 mg/kg, more preferably about 2 mg/kg to about 10 mg/kg. The number of administrations may vary, depending on the individual patient and the severity of the autoimmune condition.

The present invention is further exemplified and supported by reference to the experimental results described below.

All references cited herein are incoporated by reference as if fully set forth.

### EXAMPLE 1

α(1,3) fucosylation of carbohydrate moieties on selectin ligands is required for selectin binding and therefore, mice doubly deficient for α(1,3)-fucosyl transferase IV and VII (FT−/−) lack functional selectin ligands on endothelial cells and T cells^{1.2}. When infected with vaccinia virus (vv), FT −/1 mice do not develop viral-specific cytotoxicity, although their CD8+ T cells are capable of vigorous viral-specific proliferation and interferon-γ(IFN-γ) production. The defect in CTL killing is not a result of impaired selectin-mediated trafficking of T cells, since mice triply deficient for L-, P- and E-selectins³ develop normal antiviral cytotoxicity. Soluble recombinant P selectin glycoprotein-1 (rec-PSGL-1) and PSGL-1 monoclonal antibody, 2PH-1³ partially block the generation of effector CTL from primed wild type T cells in vitro. These results suggest that the killer function of antigen-specific CD8+ T cells develops independently of their ability to proliferate and secrete cytokines and critically depends on a α(1,3)-fucosylated PSGL-1 related molecule.

Selectins and their ligands are surface molecules reciprocally expressed on endothelial cells and leukocytes, which through their interactions initiate leukocyte rolling, the first step required for leukocyte migration through the vascular endothelium⁶⁻⁸. The lectin domain of selectins is recognized by sialyl Lewis x (sLex) related carbohydrates presented on cellular protein scaffolds and the oligosaccharide modifications on sLex moieties by glycosylation, sialylation, fucosylation and sulfation determine the fine specificity of the selectin-ligand interaction⁹⁻¹³. The central importance of fucosylation for selectin binding was shown in FT IV and VII-doubly deficient mice where L-, P- as well as E-selectin mediated leukocyte rolling is severely compromised and results in an impaired DTH response to peripheral antigen challenge^{1.2}. How defective selectin ligand function affects systemic antigen recognition is not known.

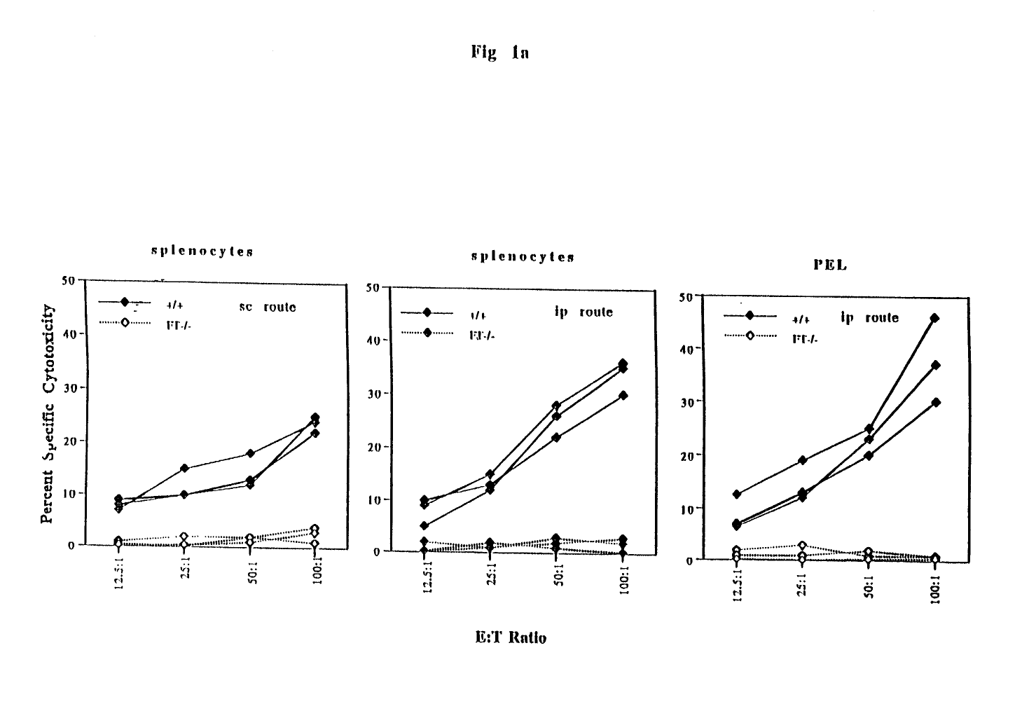

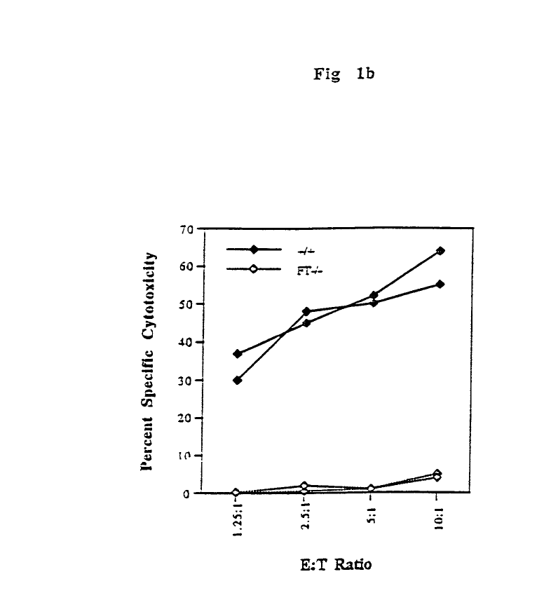

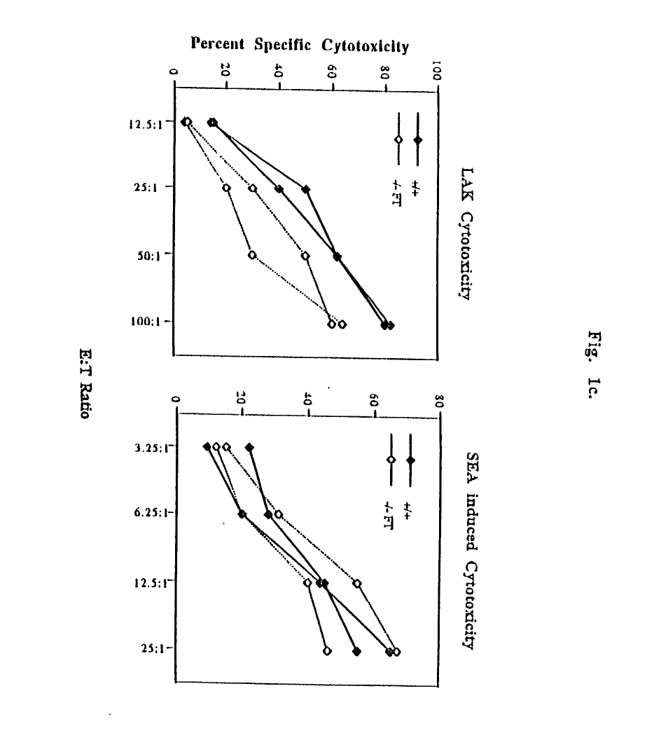

Vaccinia virus induces an acute infection in mice resulting in the generation of a robust T cell mediated immune response and viral-specific cytotoxicity can be demonstrated directly from freshly isolated splenocytes and PEL without restimulation in vitro¹⁴. Thus, vv infection provides a convenient acute infection model to study the generation of T cell response in vivo. We studied the T cell response of FT−/−mice using this model. Wild type and FT−/−mice were infected with vv via a peripheral (subcutaneously at base of the tail (sc)) or a systemic route (intraperitonially (Ip)) and viral-specific cytotoxicity was assessed using peritoneal exudate lymphocytes (PEL) and/pr splenocytes obtained on day 10 (sc route) or 7-(1p route) post infection (pf). Wild type mice showed high levels of cytotoxicity, whereas splenocytes and PEL from FT−/−mice exhibited no detectable cytotoxicity, irrespective of the route of infection (FIG. 1a). To determine the extent of the defective DTL response, we tried to enrich for viral-specific CTL by stimulating primed splenocytes (obtained 7 days pi) with vv in vitro. CTL activity was assessed after 5-7 days of culture. Although equal number of large cells could be detected in wild type but not FT−/−culture (FIG. 1b). Similar results were obtained with wild type or FT−/−lung fibroblast target cells (data not shown) indicating that these observations are not consequent to a peculiarity of the MC57G target cells used in the earlier assays. These results if the killing ability of FT−/−T cells is globally defective or, is restricted to viral-specific killing, we tested lymphocyte activated killer (LAK) function and Staphylococcal enterotoxin A (SEA) induced CTL activity in vitro. In both assays, the killer function in FT−/−animals was not severely compromised (FIG. 1c). Thus, there is a profound and specific defect in the generation of Class [-restricted antigen-specific effector CTA in the FT−/−animals.

In addition to a strong CTL response, vaccinia infection elicits natural killer (NK) cell function and γ interferon production by NK cells, CD4+ and CD8+ T cells as well as a strong humoral immune response¹⁵. Although CD8+ CTL response may be a major mediator of protection in normal animals¹⁶¹⁷, mice lacking CD8+ T cells as well as mice deficient in an important component of CTL machinery, perforin are able to clear vv suggesting that NK cells function, γ-interferon secretion and normal antibody response can compensate for the lack of anti-viral CTL^{15,18-20}. These parameters are not defective in FT−/−mice (not shown). Accordingly, although grossly defective in the generation of anti-viral CTL, FT−/−mice could clear vv similar to wild type mice (not shown). These results indicate that α(1,3)-fucosyl transferase deficiency selectively affects the generation of effector CTL. We further analyzed these mice to clarify the reasons for their defective CTL response.

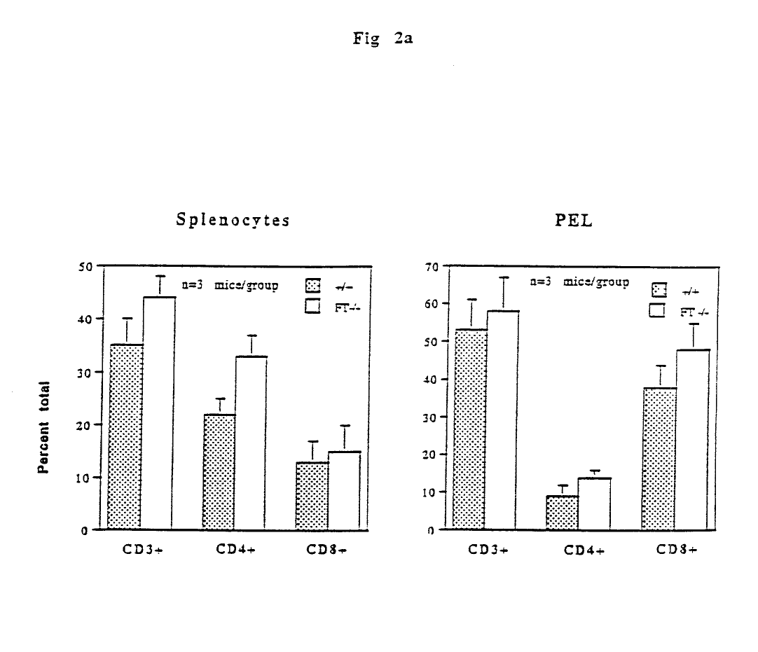

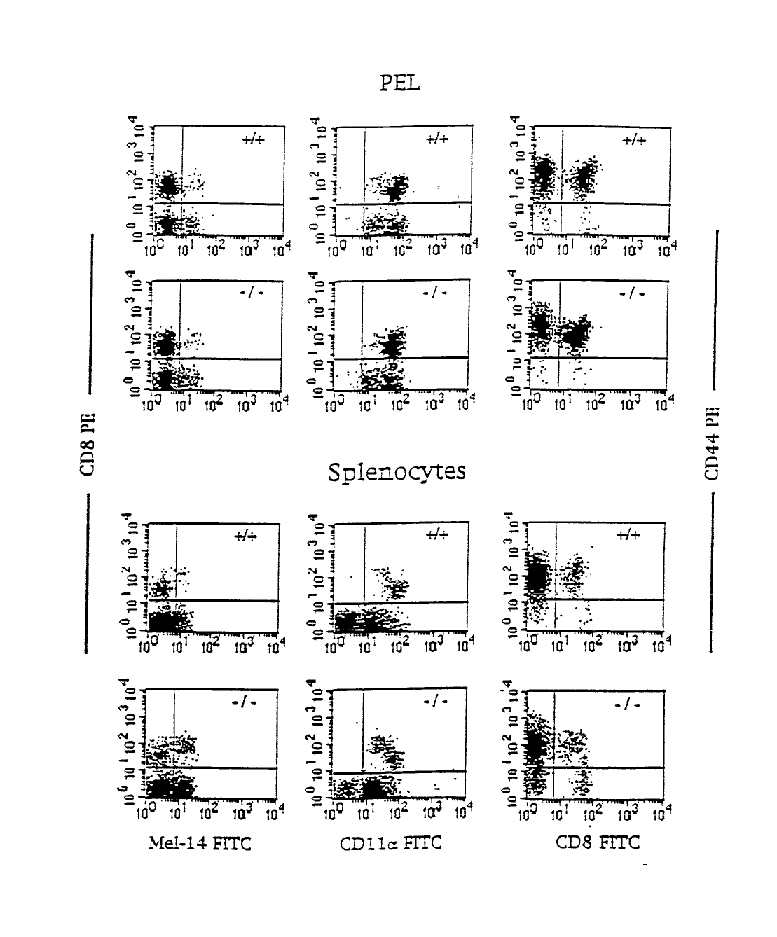

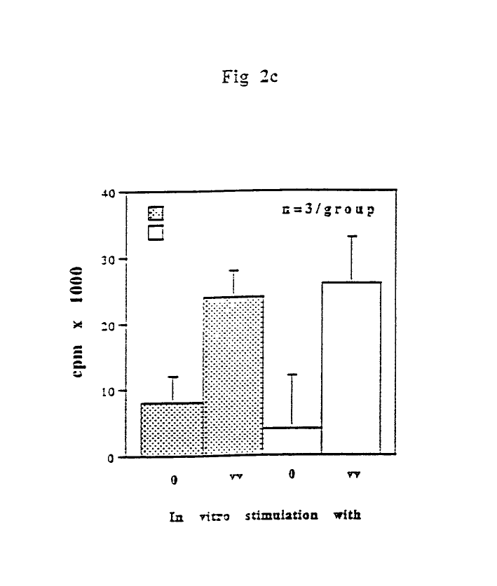

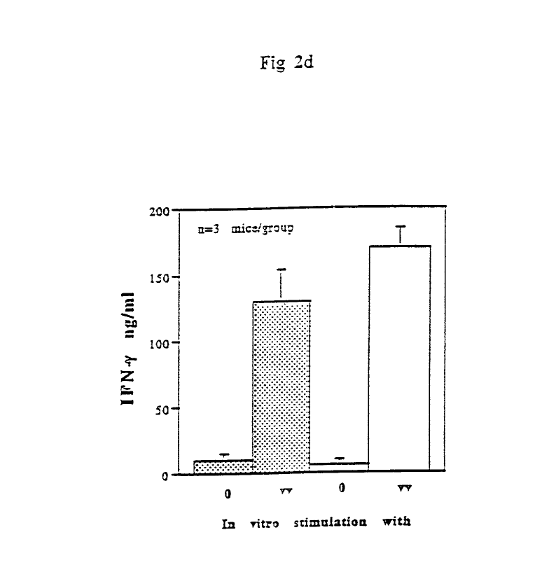

FT−/−mice are severely compromised for lymphocyte homing to peripheral lymph nodes^{1,21}, suggesting the possibility that failure to find anti-viral CTL in the FT−/−mice may be due to defective T cell priming in the peripheral of visceral lymph nodes, leading in turn to diminished levels of vv-specific CTL in the spleen. It was also possible that the PEL from FT−/−mice had no detectable CTL because of diminished T cell trafficking into the peritoneal cavity. To address these possibilities, we compared splenocytes and PEL from wild type and FT−/−mice for T cell subset representation, and for their activation status. Splenocytes and PEL from both wild type and FT−/−mice had comparable proportions of CD4+ and CD8+ T cells (FIG. 2a). Moreover, CD4+ and CD8+ T cells are in both PEL and the spleen exhibited similar levels of L-selectin, LFA-1 and CD44 (FIG. 2b). The absolute numbers of cells recovered from the peritoneal cavity was reduced on an average by 50% in the FT−/−mice compared to wild type mice, suggesting some defect in trafficking of cells into the peritoneal cavity. This however, can not explain the defective CTL function in the FT−/−mice since total cell numbers are equalized to that in wild type mice in CTL assays to determine the E:T ratios. Thus, although similar numbers of activated CD8+ T cells were tested in the CTL assays, viral-specific cytotoxicity was not detected in FT−/−mice. These results imply that in the FT−/−mice, CD8+ cells in the spleen and PEL are activated but are not able to mediate cytolytic function.

During an inflammatory condition like a viral infection, in addition to antigen-specific cells, non-specific T cells may be activated and traffic to the site of invention^{22,23}. However, recent data using TCR transgenic mice and MHC-peptide tetramers indicate that most activated cells are indeed antigen-specific²⁴⁻²⁶. To determine whether the activated CD8+ T cells in the spleen and PEL are antigen-specific, cells from infected mice were immunomagnetically depleted of CD4+ T cells and NK cells, and tested for vv-specific proliferation. Both wild type and FT−/−CD8+ T cells proliferated comparably and specifically in response to vv stimulation (FIG. 2c). Since IL-2 production is required for T cell proliferation, this result suggested that cytokine production may not be defective in FT−/−CD8+ T cells. We also assayed for vv-stimulated production of the major CD8+ T cell cytokine, interferon-γ production was comparable in wild type and FT−/−CD8+ T cells (FIG. 2d). These results suggest that the generation of viral-specific CD8+ T cells, their viral-specific proliferation and cytokine release are not altered in the FT−/−mice.

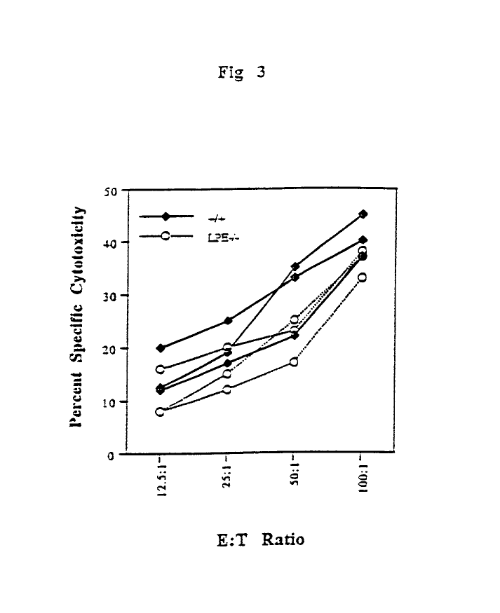

To determine whether the absence of effector CTL in FT−/−mice is a result of defective selectin or selectin ligand function, we tested mice triply deficient for L-, P- and E-selectins for their ability to generate antiviral CTL after vv infection. Triple selectin deficient mice, like wild type mice and unlike FT−/−mice, exhibited a robust CTL activity (FIG. 3). Thus, the defect in effector CTL generation in the FT−/−mice is unrelated to a defective selectin function but is a consequence of a selectin ligand function.

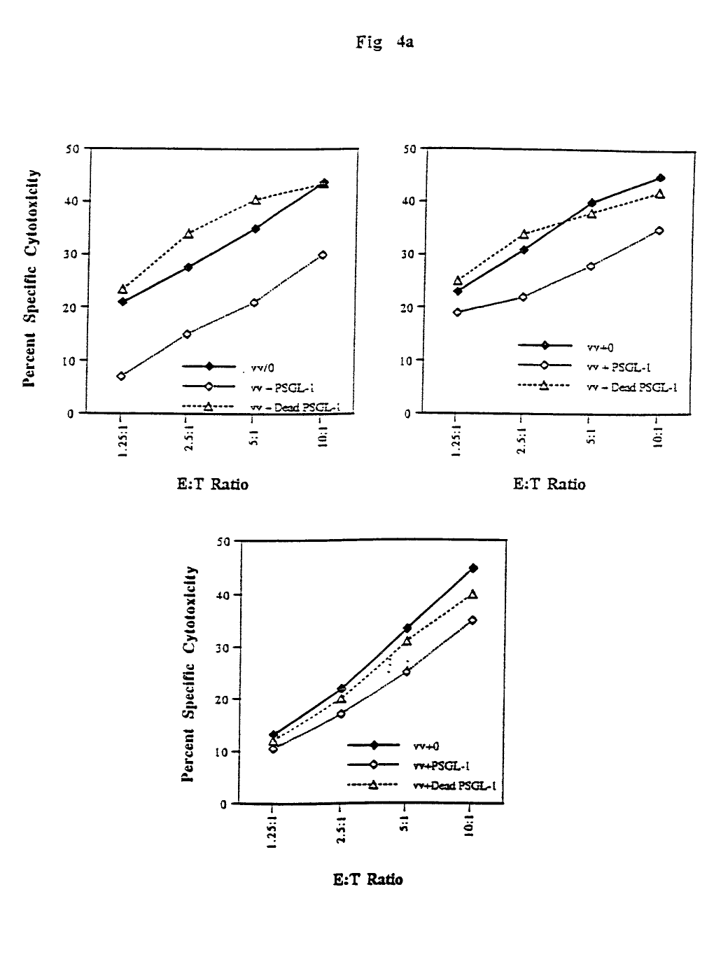

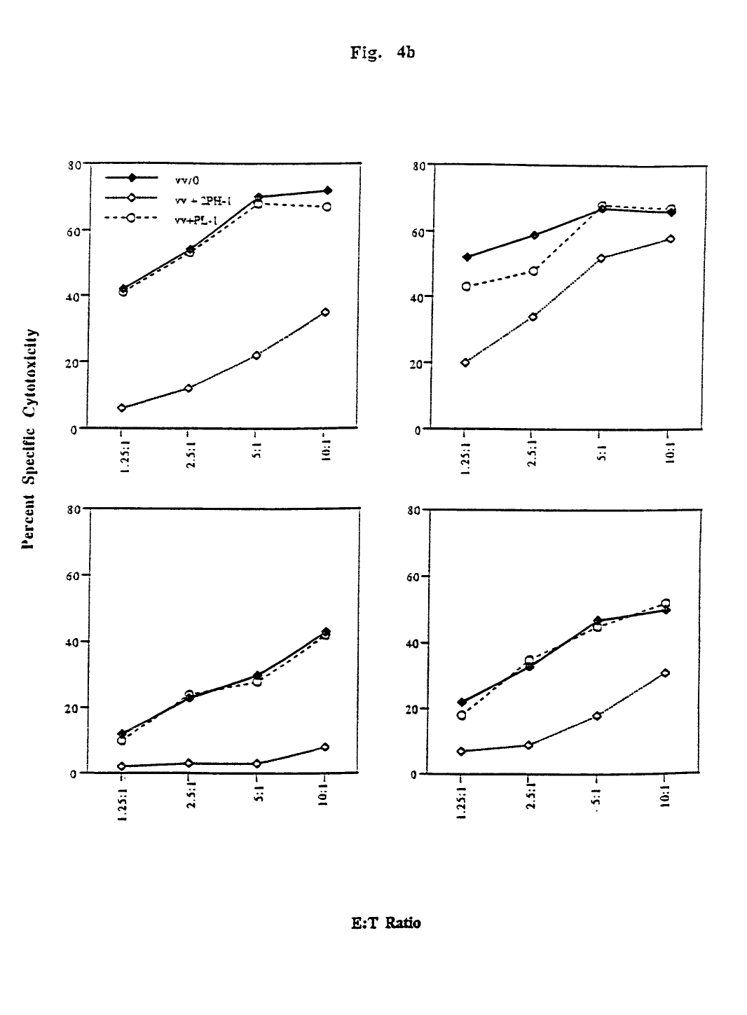

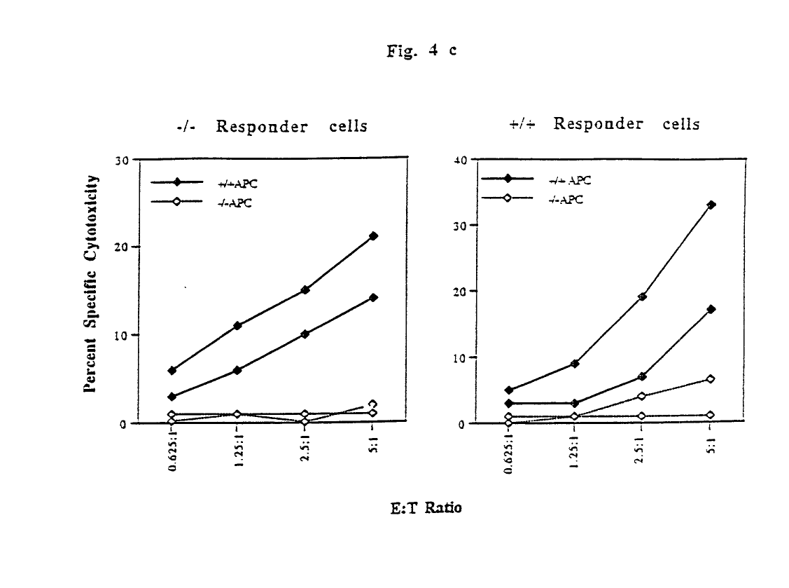

Collectively our results indicate that the cytotoxic production is impaired in FT−/−mice and that an α(1,3)-fucosylation defect in FT−/−mice could account for the lack of CTL effector function. We therefore reasoned that an Fuc-T-dependent fucosylated structure on either T cells or antigen presenting cells (APC) might be required for the generation/mediation of CTL effector function. PSGL-1 is a prominent α(1,3)-fucosylated glycoprotein expressed on APC and T cells²⁷. This molecule is functionally deficient in FT−/−mice^{1.2}, and represents one candidate for a Fuc-T-dependent molecule required for CTL activation. Thus, we investigated the effect of soluble recombinant PSGL-1 and of PSGL-1 function blocking antibody, 2PH-1 on secondary in vitro stimulation of primed viral-specific CD8+ T cells derived from wild type mice. Wile type mice were infected with vv and on day 7 pi, their splenocytes were stimulated in vitro with vv in the absence or presence of either soluble PSGL-1 or its non-fucosylated mutant and, or PSGL-1 blocking monoclonal antibody (2PH-1)⁵ or control antibodies (anti-L selectin Mel 14, or anti-human PSGL-1 antibody PL-1²⁸) Both soluble PSGL-1 and function blocking anti-murine PSGL-1 antibody, but not non-fucosylated soluble PSGL-1 or control antibodies tested partially inhibited development of viral-specific CTL relative to control antibodies (FIG. 4a) 4b and data not shown). However, neither soluble PSGL-1 nor anti-murine PSGL-1 antibody had an inhibitory effect when added during the CTL assay (not shown). Thus, α(1,3)-fucosylated PSGL or a closely related molecule appears to be required for the generation of functional CTL but not for target cell lysis.

To determine if this fucosylated molecule is required on APC or on T cells, we asked if wild type APC could activate lytic function in vv-primed FT−/−CD8+ T cells, or if FT−/−APC were defective in their ability to activate CTL from primed wild type CD8+ T cells. Wild type and FT−/−mice were infected with vv and on day 7 pi, splenic CD8+ T cells were selected and stimulated with T cell depleted, vv-infected, γ-irradiated wild type of FT−/−splenocytes. Cytolytic function was detected in both wild type and FT−/−CD8+ cells when stimulated with wild type APC, whereas FT−/−APC were incapable of eleciting CTL activity on CD8+ cells from either wild type or FT−/−mice (FIG. 4c). Thus, a fucosylated molecule similar to PSGL-1, and expressed by APC appears to be required for effector CTL generation.

Taken together, our results suggest that APC-CD8 T cell interaction through an α(1,3)-fucosylated molecule is necessary for the development of antigen-specific CD8 CTL effector function but is not required for antigen-specific CD8 T cell proliferation or cytokine secretion. The fact that anti-murine PSGL-1 as well as soluble PSGL-1 inhibited effector function generation by wild type CD8+ T cells and that a similar defect was not seen in selectin-deficient mice suggests that PSGL-1 recognition of a counter receptor(s) that is (are) distinct from selectins is (are) required. Although PSGL-1 was originally identified as the ligand for P selectin, it is now clear that carbohydrate modifications have profound effects on its binding. Activated Ta1 cells, but not resting T cells or activated Th-2 cells, bind P-selectin, although PSGL-1 is expressed in equivalent amounts of all of these cell types²⁹. Carbohydrate modifications which confer binding ability to HECA 452, an antibody directed against the cutaneous lymphocyte antigen (CLA), modulate PSGL-1 binding to E-selectin³⁰. Our results raise the possibility of additional, selectin independent receptor(s) for PSGL-1. Identification of the receptors will likely lead to insights into the mechanism of effector CTL generation and might provide tools to modify CTL killer function selectively, either to enhance it for viral infections and tumors or, to suppress it in autoimmune diseases.

Figure Legend—FIG. 1

FT−/−mice are severely compromised in generating viral-specific effector CTL, but have virtually normal LAK and SEA induced CTL activity. 1a. Splenocytes from wild type or FT−/−mice infected with vv sc, and Splenocytes & PEL from mice infected ip were tested for cytolysis of vv infected MC57G (H2b) targets by 4 h Cr release assay. 1b. Splenocytes from ip infected mice were restimulated in vitro by incubation with vv infected autologous splenocytes for 5 days and tested for antiviral cytoxicity. For all the assays, background killing of uninfected MC57G targets (which was <3%) was subtracted to calculate % specific killing. 1c. Splenocytes were cultured in vitro for 3 days in the presence of either 200 IU/ml recombinant IL-S and tested for lysis of Yac-1 target cells (LAK activity) or in the presence of 10 μg/ml SEA and CD8+ cells were selected and tested for lysis of Raji cells coated with SEA.

Figure Legend—FIG. 2

FT−/−mice generate activated CD8+ T cells which proliferate and produce cytokines in a viral-specific manner. Splenocytes and PEL from vv infected mice stained with FTTC-conjugated and mouse Thy 1.2, CD4 or CD8 monoclonal antibodies (2a) or doubly stained with CD8 FTTC or PE and CD62-L FTTC, CD11α FTTC or CD44 PE (2b) were analyzed by flow cytometry. For 2c and d, splenocytes from vv infected mice were immunomagnetically depleted of CD4+ T cells and NK cells and stimulated with vv as described in FIG. 1c. Three days later, culture supernatants were tested for IFN-γ levels (2c) and cells were pulsed with ³H thymidine for 3 h and counted for 3 H incorporation (2d). Shown is the average +/−SEM or 3 pairs of mice.

Figure Legend—FIG. 3

FT−/− but not selectin−/−mice fail to generate viral-specific effector CTL. Wile type mice and mice deficient for L-, P-, and -E selectins were infected with vv and their splenocytes were tested for antiviral cytotoxicity on day 7 pi as described in FIG. 1.

Figure Legend—FIG. 4

Soluble PSGL-1 and anti-murine PSGL-1 antibody inhibits development of effector CTL by primed wild type CD8+ T cells in vitro. Splenocytes harvested from wild type mice on day 7 post vaccinia infection were stimulated with vv in the absence or presence (20 μg/ml) of soluble recombinant PSGL-1 or non fucosylated PSGL-1 (dead PSGL-1) (4a) or of anti-murine PSGL-1 antibody, 2 PH-1, or anti-human PSGL-1 antibody, PL-1 (4b). Viral-specific cytotoxicity was measured 5 days later. 4c. FT−/−APC abrogates and wild type APC restores effector CTL generation. Wild type and FT−/−mice were infected with vv and on day 7 pi, CD8+ T cells (responders) were positively selected and stimulated with vv infected and γ-irradiated wild type or FT−/−APC (T cells depleted splenocytes). Viral-specific cytotoxicity was assayed 5 days later.

Methods

Vaccinia viral infection. FT IV and VII−/−, L-, P- and E-selectin−/−mice and their wild type counterparts were maintained under SPF facility at the Center for Blood Research. Mice 6-8 weeks of age and matched for sex were used for the studies. Mice were infected with WR strain of vv (ATCC) either sc at the base of the tail or lp (10⁵ pfu/mice in 0.2 ml PBS).

Cytotoxicity assays. To test viral-specific cytotoxicity, on day 7 pi, peritoneal exudate cells were harvested by flushing with 3 mls of PBS and/or spleens were collected. Splenocytes and PEL were depleted of RBC by lysis in 0.17 M ammonium chloride and the cells were tested for killing of ⁵¹Cr labeled, MC57G targets uninfected or infected with vv as described earlier³¹. For LAK assay, splenocytes from normal mice were cultured in the presence of 200 IU/ml recombinant IL2, and 3 days later, cells were tested for killing of ⁵¹Cr labeled Yac-1 targets. For SEA induced cytotoxicity assay, splenocytes were cultured in the presence of 10 μg/ml SEA (Sigma) and CD8 T cells were selected (see later) and tested for lysis of Raji cells coated with SEA (100 ng/ml for 30 min before the assay). Cytotoxicity was defined as (test release-spontaneous release)/(maximum release-spontaneous release)×100. Percent killing of uninfected targets (vv cytotoxicity) or uncoated target (SEA induced cytoxicity) was subtracted from that of infected/coated targets to calculate viral-specific cytotoxicity.

Antibody staining, flow cytometry and immunomagnetic depletion. To determine T cell subset numbers, splenocytes and PEL were stained singly with FTTC-conjugated anti-mouse CD3, CD4 or CD8 monoclonal antibodies (Pharmingen). Activated CD8+ T cells defined as L-selectin low, LFA-1 high and CD44 high, were assayed by dual staining with PE CD8 X FTTC Mel-14, FTTC CD11α or FTTC CD8 X PE CD44 (Pharmingen). For depletion of CD4+ T cells and NK cells, cells were stained with purified rat anti-mouse CD4 and NK 1.1 antibodies, washed and incubated with goat anti-rat Ig G coated magnetic beads (Dunai, 10 beads/cell). The depleted population contained <3% CD4 or NK cells as determined by flow cytometry.

In vitro restimulation with vv. For APC, splenocytes harvested 6-7 days post vaccinia were depleted of T cells using anti-CD3 coated Dynal beads and infected with vv (10 pfu/cell, 2 h at 37° C.), irradiated (400 rads) and UV-treated as described in³². 5×10⁶ infected cells were cultured with 5×10⁶ autologous uninfected splenocytes in 24 well culture plates for 4-5 days before testing for CTL activity. In some experiments, CD4+ T cells and NK cells were depleted as described above. In some other experiments, CD8+ cells were positively selected using CD8+ milteny beads according to manufacturer's instructions. In some experiments, at the time of in vitro stimulation, soluble recombinant PSGL-1 Ig chimera, its non-fucosylated variant (dead PSGL-1) (gifts of Genetics Institute, Cambridge, Mass.), anti-murine PSGL-1 antibody, 2 PH-1, anti-human PSGL-1 antibody, PL-1 (gift of . . . ), and anti-murine L-selectin antibody, Mel-14 (gift of . . . ) were added at a final concentration of 20 μg/ml.

Lymphocyte proliferation and IFN-γ assay. 2×10⁵ splenocytes, depleted of CD4+ T cells and NK cells as described above, were cultured with equal numbers of γ-irradiated splenocytes that were uninfected or infected with vv in triplicate wells of 96 well trays. Three days after stimulation, 50 μg supernatants were harvested for IFN-γ assay and the cultures were pulsed with ³H thymidine (0.5 μCl/well) for 6-8 h, harvested and counted for 3H incorporation as described in²⁷. Supernatants were assayed for IFN-γ using IFN-γ mini assay kit (Antigen, MA, USA) calibrated with and IFN-γ standard according to manufacturers protocol.

### REFERENCES

1. May, P., Tail, A. D., Petryniak, B., Rogers, C. E., Smith, P. L., Marks, R. M., Kelly, R. J., Gersten, K. M., Cheng, G., Saunders, T. L., Camper, S. A., Camphausen, R. T., Sullivan, F. X., Isogai, Y., Hindsgaul, O., von Andrian & Lowe, J. B. The α(1,3) Fucosyltransferase Fuc-TVII controls leukocyte trafficking through an essential role in L-, E-, and P-selectin biosynthesis. Cell 86, 643-653 (1996).

2. Unpublished data?

3. LPE selectin KO.

4. Takada, M. K., Nadeau, K. C., Shaw, G. D., Marquette, K. A. & Tilney. The cytokine-adhesion molecule cascade in ischemia/reperfusion injury of the rat kidney. Inhibition by a soluble P-selectin ligand. J. Clin. Invest. 99, 2682-2690 (1997).

5. Borges, E., Eytner, R., Moll, T., Steegmaier, M., Matthew, A., Campbel, L. P., Ley, K., Mossmann, H. & Vestweber, D. The P-selectin glycoprotein ligand-l is important for recruitment of neutrophils into inflamed mouse peritoneum. Blood 90, 1934-1942 (1997).

6. Butcher, E. C. Leokocyte endothelial cell recognition: Three (or more) steps to specificity and diversity. Cell 67, 1033-1036 (1991).

7. Picker, L. J. Control of lymphocyte homing. Curr. Opin. Immunol. 6, 394-406 (1994).

8. Springer, T. A. Traffic signals for lymphocyte recirculation and leukocyte emigration: The multi-step paradigm. Cell 76, 301-314 (1994).

9. Hemmerich, S., Leffler, H. & Rosen, S. D. Structure of the O-Glycans in GlyCAM-1, an endothelial derived ligand for L-selectin. J. Biol. Chem. 270, 12035-12047 (1995).

10. Moore, K. L., Eaton, S. F., Lyons, D. E., Lichenstein, H. S., Cummings, R. D. & McEver, R. P. The P-selectin glycoprotein ligand from human neutrophils displays sialated, fucosylated, o-linked poly-N-acetyllactosamine. J. Biol. Chem. 269, 23318-23327 (1994).

11. Sako, D., Comess, K. M., Barone, K. M., Camphausen, R. T., Cumming, D. A., and Shaw, G. D. A sulfated peptide segment at the amino terminus of PSGL-1 is critical for P-selectin binding. Cell 83, 323-331 (1995).

12. Poutyani, T. & Seed, B. PSGL-1 recognition of P-selectin is controlled by a tyrosine sulfation consensus at the PSGL-1 amino terminus. Cell 83, 333-343 (1995).

13. Li, F., Wilkins, P. P., Crawley, S., Weinstein, J., Cummings R. D. & McEver, R. P. Post-translational modifications of recombinant P-selectin glycoprotein ligand-1 required for binding to P- and E-selectin. J. Biol. Chem. 271, 3255-3264 (1996).

14. Bennink, J. R., & Yewdell, J. W. Recombinant vaccinia viruses as vectors for studying T lymphocyte specificity and function. Curr. Topics Microbiol. Immunol. 163, 153-184 (1990).

15. Spriggs, M. K., Koller, B. H., Sato, T., Crissey, P. J., Fanslow, W. C., Smithies, O., Voice, R. F., Widmer, M. B. & Maliszewski, C. R. β2-microglobulin-CD8+ T cell-deficient mice survive inoculated with high doses of vaccinia virus and exhibit alter IgG responses. Pros. Natl. Acad. Sci. USA 89, 6070-6074 (1992)

16. Binder, D., & Kundig, T. M. Antiviral Protection by CD8+ versus CD4+ T cells: CD8+ T cells correlating cytotoxicity in vitro are more efficient in anti-vacccinia protection than CD4+ dependent interleukins. J. Immunol. 146, 4301-4307 (1991).

17. Blanden, R. V. Mechanisms of recovery from a generalized viral infection: mousepox. 3. Regression of invectios foci. J. Exp Med. 133, 1091104 (1971).

18. Kagi, D., Seiler, P., Pavlovic, J. Burki, K., Zinkemagal, R. M. & Hengartner, H. The roles of perforin- and Fas-dependent cytotoxity in protection against cytopathic and noncytopathic viruses. Eur. J. Immunol. 25, 3256-3262 (1995).

19. Muller, U., Steinhoff, U., Reis, L. F. L., Hemmi, S., Pavlovic, J., Zinkeragel, R. M., & Aguet, M. Functional rope of type I and type II interferons in antiviral defense. Science 264, 1918-1921 (1994).

20. Kagi, D., & Hengartner, H. Different roles for cytotoxic T cells in the control of infections with cytopathic versus noncytopathic viruses.

21. FT−/− T cells are severely compromised in homing to PLN—Intravital data published?

22. Tripp, R. A., Hou, S., McMickle, A., Houston, J. & Doherty, P. C. Recruitment and proliferation of CD8 T cells in respiratory virus infections. J. Immunol. 154, 6013-6021 (1995).

23. Tough, D. F., Borrow, P. & Sprent, J. Induction of bystander T cell proliferation by viruses and type 1 interferon in vivo. Science 272,1947-1950 (1996).

24. Butz, E. A. & Bevan, M. J. Massive expansion of antigen-specific CD8 T cells during an acute virus infection. Immunity 3, 167-175 (1998).

25. Counting antigen-specific CD8 T cells: A reevaluation of bystander activation during viral infection. Immunity 8, 177-187 (1998).

26. Ogg, G. S., Jin, X., Bonhoeffer, S., Dunbar, P. R., Nowak, M. A., Monard, S., Segal, J. P., Cao, Y., Rowland-Jones, S. L., Cerundolo, V., Hurley, A., Markowitz, M., Ho, D. D., Nixon, D. F. & McMichael, A. J. Quantitation of HIV-1 -specific cytotoxic t lymphocytes and plasma load of viral RNA. Science 279, 2103-2106 (1998).

27. Laszik, Z. P., Jansen, P. J., Cummings, R. D., Tedder, T. F., Mcever, R. P. & Moore, K. L. P-selectin ligand glycoprotein ligand-1 is broadly expressed in cells by myeloid, lymphoid and dentritic lineage and in some nonhematopoietic cells. Blood 88, 3010-21 (1996).

28. Norman, K. E., Moore, K. L., Mcever, R. P. & Ley, K. Leucotyte rolling in vivo is mediated by P-selectin glycoprotein-1. Blood 86, 4417-1421 (1995).

29. Borges, E., Tietz, W., Steegmaier, M., Moil, T., Hallmann, R., Hamann, A., & Vestweber, D. P-selectin glycoprotein-1 (PSGL-1) on T helper 1 but not on T helper 2 cells bind to P-selebtin and and supports migration into inflammed skin. J. Exp. Med. 185, 573-578 (1997).

30. Fuhlbrigge, R. C., Kierfer, J. D., Armerding, D. & Kupper, T. S. Cutaneous lymphocyte antigen is a specialized form of PSGL-1 expressed on skin-homing T cells. Nature 389, 978-981 (1997).

31. Manjunath, N., Correa, M., Ardman, M. & Ardman, B. Negative regulation of T lymphocyte activation and adhesion by CD43. Nature 377, 535-538 (1995).

32. Shankar, P., Fabry, J. & Liberman, J. A simple method to selectively expand HIV-1 cytotoxic T lymphocytes in vitro. J. Immunol. Invest. 24, 489-497 (1995).

### EXAMPLE

Mice that are doubly deficient in the α(1,3)-fucosyltransferases, FT-IV and FT-VII (FT−/−mice), lack functional selectin ligands on leukocytes and endothelial cells. Here, we studied the effect of FT deficiency on CD8+ T cell responses to vaccinia virus infection. FT−/−mice developed markedly fewer cytotoxic T cells as compared to wild-type mice, although comparable numbers of CD8+ T cells accumulated at the site of infection in both strains and were capable of vigorous viral-specific proliferation. This defect in CTL generation was not due to impaired selectin-dependent T cell trafficking, because mice triply deficient in L-, P- and E-selectin developed normal antiviral cytotoxicity. Coincubation with wild-type APC induced CTL activity in primed CD8⁺ T cells from both FT−/−and wild-type mice, whereas FT−/−APC did not induce CTL generation in either strain. CTL generation by wild-type APC was inhibited by anti-P-selectin glycoprotein ligand (PSGL)-1 and by coincubation with α(1,3)-fucosylated PSGL-1/Ig chimera, whereas non-fucosylated PSGL-1/Ig had no effect. These results suggest a novel function for PSGL-1 and perhaps other fucosylated molecules on APC in the generation of CTLs from antigen-specific CD8+ T cells, which is distinct from their ability to bind selecting Cytotoxic T lymphocytes (CTL) are critical mediators of antigen-specific host defense against viral infections. Before a CTL response can be mounted, naive CD8+ T cells must first encounter viral antigen on professional antigen-presenting cells (APCs) in secondary lymphoid organs. Antigen-activated T cells proliferate for several days and eventually migrate to the site of viral infection. Finally, they acquire effector functions, namely the ability to kill other cells that express cognate antigen on MHC class I and to produce effector cytokines, particularly interferon (IFN)-γ. The CTL response is thus dependent on the targeted movement (homing) of leukocytes in the intra- and extravascular compartments. Antigen-laden APC must initially migrate from the site of infection to organized lymphoid tissues. Here, they stimulate naive T cells, which home to these organs from the blood. Subsequently, activated T cells must find their way back into the blood stream and from there into infected peripheral tissues.

Leukocyte migration to many lymphoid and non-lymphoid organs requires the concerted action of one or more of the three selectins (L-, E- and P-selectin, CD62) and their ligands, which are reciprocally expressed on endothelial cells and leukocytes (1-3). Selectins mediate leukocyte rolling in microvessels by binding to sialyl-Lewis^{X} (sLeX) and related carbohydrates that are frequently presented on sialomucin scaffolds such as PSGL-1 (4, 5). A critical aspect of selectin-binding carbohydrates is α(1,3)-fucosylation of one or more N-acetyl-glucosamine residues in sialylated core-2 glycans. So far, five different α(1,3)-fucosyltransferases (FTs) have been identified in mammals, but only FT-IV and FT-VII are expressed by leukocytes and endothelial cells (6). Mice that are deficient in FT-VII have a defect in selectin-dependent leukocyte rolling and migration to sites of acute inflammation and lymphocyte homing to lymph nodes is markedly reduced (7). In contrast, FT-IV−/−mice have only a mild defect in leukocyte rolling, whereas FT-IV+VII doubly deficient (FT−/−) mice have a phenotype more severe than that of FT-VII−/−animals (8).

The importance of the selectins has been documented in many settings, including acute inflammation, atherosclerosis and cutaneous hypersensitivity responses to peripheral antigen challenge (reviewed in 2, 4, 5). Moreover, it has been reported that functional PSGL-1 is upregulated on many T cells after antigen recognition, and is required for their recruitment into the inflamed peritoneum (9). Correspondingly P- and E-Selectin antibodies severely compromise both CD4 and CD8 T cell recruitment to sites of acute inflammation in mice (9). However, how selectins and their ligands affect T cell recruitment and immune responses during a viral infection in vivo is not known. In particular, the role of these molecules during a CTL response to viral antigen challenge has not been examined. To address this question, we injected vaccinia virus intraperitoneally (i.p.) into FT−/−mice and animals that were triply deficient in L-, E- and P-selectin (selectin−/−) (10). Vaccinia virus has been shown to induce an acute infection in wild-type mice resulting in the generation of a robust T cell-mediated immune response and viral-specific cytotoxicity can be demonstrated directly from freshly isolated splenocytes and peritoneal exudate lymphocytes (PEL) without restimulation in vitro (11).

All wild-type and genetically deficient animals survived the infection and virus levels became undetectable within 10 days post infection (p.i.) indicating that selectins and carbohydrates modified by FT-IV and/or FT-VII are not essential for viral clearance. However, the immune response to vaccinia virus is multi-facetted. In addition to a strong CTL response, vaccinia infection elicits natural killer (NK) cell function and IFN-γ production by NK cells, CD4⁺ and CD8⁺ T cells as well as a strong humoral immune response (11-17). Although CD8⁺ CTL are the principal mediators of protection in normal animals (13), mice lacking CD8⁺ T cells as well as mice deficient in perforin, an important component of the CTL machinery, can clear vaccinia infections (12, 15, 17). Therefore, normal viral clearance in mice that are deficient in FTs or selectins does not exclude that these molecules have a role in the generation, migration or function of anti-viral CTL.

Thus, we analyzed the number, composition and function of peripheral blood mononuclear cells (PBMC), PEL and splenocytes obtained from wild-type and knockout mice at day 7 p.i. Selectin−/− and FT−/−mice had much higher leukocyte counts in peripheral blood and spleen than did wild-type mice (Table 1). These results are in accordance with earlier studies that have demonstrated a role for selectins in hematopoiesis and leukocyte homeostasis (7, 8, 10, 18). Although the frequency of CD4⁺ T cells in blood and spleen was comparable in all strains, CD8⁺ T cell fractions and total cell counts in these compartments were elevated in both selectin−/− and FT−/−mice. However, at the site of infection (peritoneum), leukocyte numbers were comparable and similar numbers of CD4⁺ and CD8⁺ T cells were recovered in PEL from wild type and mutant mice. CD8⁺ T cells were the most frequent subset in PEL of all strains, probably reflecting the dominance of CD8⁺ T cell response in vaccinia infection (13). We conclude that selectin-ligand interaction is not essential for T cell migration to the inflamed peritoneal cavity in this infection model.

Table 1. also shows that equivalent fractions of T cells in the blood, spleen as well as in PEL expressed activation markers (L-selectin^{Lo} and CD44^{Hi}) suggesting that antigen-specific priming of T cells can occur normally in the absence of selectins or their ligands. During an inflammatory condition like a viral infection, not only antigen-specific T cells, but also some non-specific bystander cells may be activated and traffic to the site of infection (19, 20). However, recent data using TCR transgenic mice and MHC-peptide tetramers indicate that most activated cells are indeed antigen-specific (21-23). Thus, it is likely that T cells in selectin−/− and FT−/−mice were exposed to vaccinia antigen, particularly in the spleen where selectins are not required for homing (7, 24).

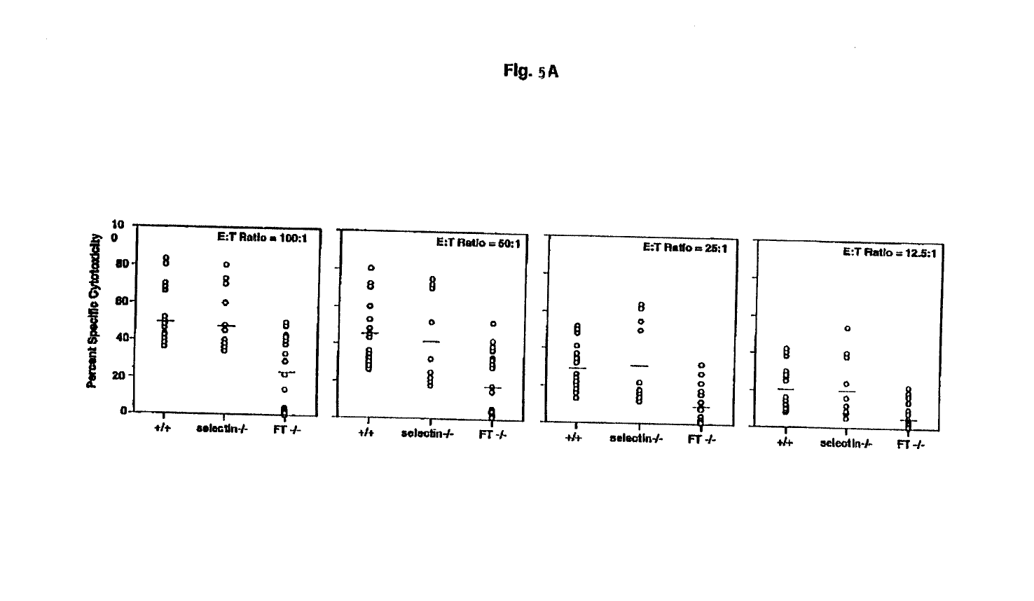

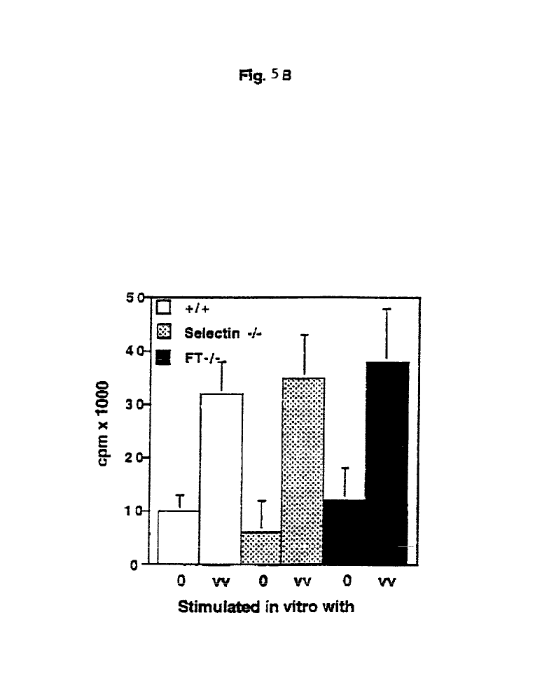

To determine to what extent the activated CD8⁺ T cells in infected animals were vaccinia-specific effector cells, we tested PEL (obtained at day 7 p.i.) of infected mice for virus-specific CTL activity (25). PEL from selectin−/−mice specifically lysed virus-infected target cells at a level that was similar to wild-type controls. In contrast, PEL T cells from FT−/−mice exhibited either markedly reduced levels of cytotoxicity (11 animals) or no detectable CTL activity (5 animals) (FIG. 5A). This observation suggested that FTs, but not selecting, may be required for the generation of anti-viral CTL activity in vivo. To determine whether this involved one enzyme or both, we also tested mice that were deficient in FT-IV or FT-VII alone. Both strains had significantly reduced CTL activity compared to wild-type mice, but the reduction was more notable in the FT-VII−/− than in the FT-IV−/−mice (not shown). The most striking reduction of CTL activity was seen in the FT-IV/FT-VII doubly deficient mice suggesting that both enzymes may be necessary to elicit optimal CTL activity. In additional experiments, we also tested mice that were singly deficient in P- or L-selectin (26, 27) or doubly deficient in P- and E-selectin (18). Vaccinia-specific CTL activity was comparable to wild-type controls in all of these strains, which were each derived from independent ES cell clones (data not shown).

Since compromised lymphocyte trafficking seemed an unlikely explanation for the surprising diminishment of CTL in FT−/−mice, we explored two alternative hypotheses. First, FT−/−T cells might be incapable of detecting or responding to vaccinia antigen. Alternatively, antigen-specific FT−/−T cells might exist and get activated, but they may not be able to kill target cells. To test whether activated CD8⁺ T cells in FT−/−mice recognize and respond to vaccinia-derived antigens, splenocytes were immunomagnetically depleted of CD4⁺T cells and NK cells, and tested for vaccinia virus-specific proliferation. CD8⁺ T cells from primed mice proliferated rapidly and specifically upon antigen challenge (FIG. 5B). There was no difference between CD8⁺ T cells from FT−/−mice compared to cells from selectin−/− or WT animals. Thus, FTs are not required for the proliferative T cell response to antigen, but may be necessary later when activated CD8⁺ T cells give rise to effector CTL.

In a separate study, we have shown that CTL activity of vaccinia-specific CD8⁺ T cells is tightly linked to the cells' ability to produce IFN-γ in response to TCR engagement (28). Indeed, when primed FT−/−CD8⁺ cells were treated with anti-CD3, they generated markedly reduced amounts of this effector cytokine compared to wild-type and selectin−/−CD8⁺ cells that were stimulated in parallel (FIG. 5C). Interestingly, IFN-γ production was also reduced in FT−/−CD4⁺ cells indicating that FT deficiency may not only affect the CD8⁺ subset (data not shown). Thus, FT−/−CD8⁺ cells lacked at least two distinct qualities of effector cells; CTL activity and IFN-γ production. These findings led us to hypothesize that FTs might be required to trigger one or more decisive events that must occur before activated T cells can give rise to differentiated effector cells.

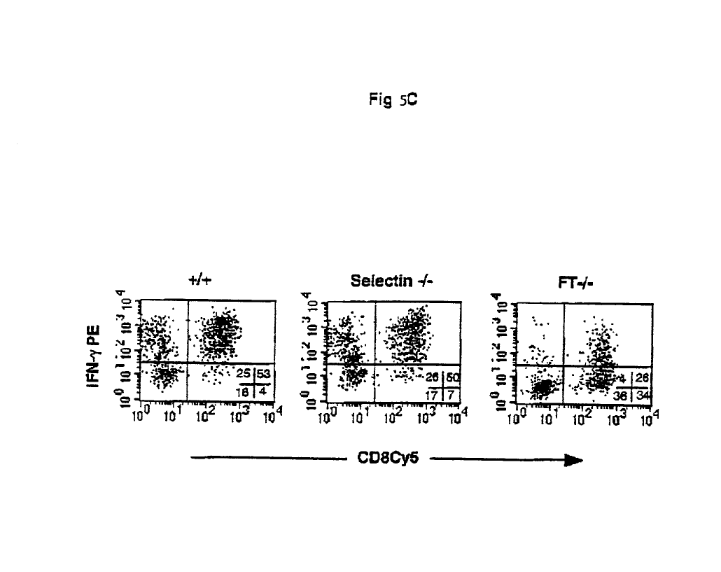

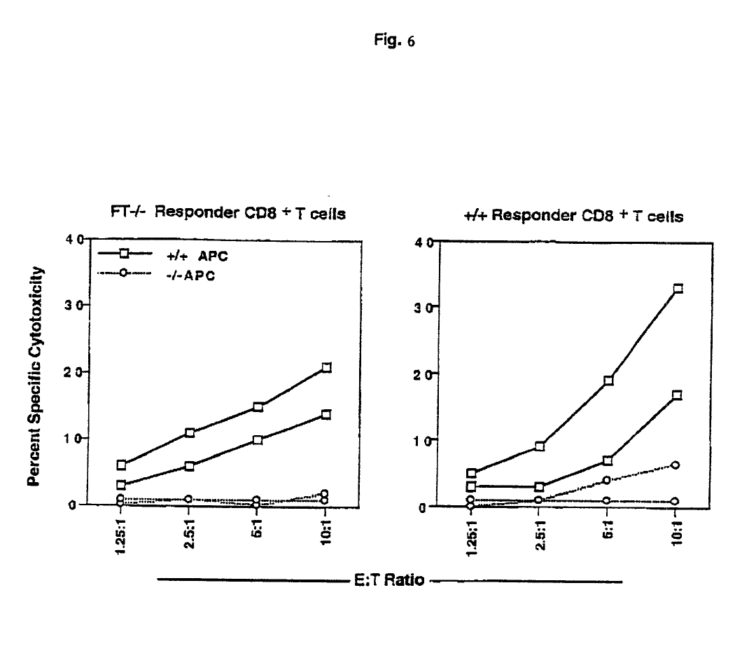

The generation of Class I-restricted CTL requires interaction of CD8⁺ T cells with APC. Thus, we asked whether FTs are required in T cells or in APC to promote CTL differentiation. We restimulated purified primed T cells from wild-type mice with APC (i.e. T cell-depleted, vaccinia virus-infected, γ-irradiated splenocytes) from FT−/−animals and vice versa (29). Cytolytic activity was reproducibly induced in both wild-type and FT−/−T cells that encountered vaccinia antigen presented by wild-type APC, whereas FT−/−APC were incapable of eliciting CTL activity on CD8⁺ cells from either wild-type or FT−/−mice (FIG. 6).

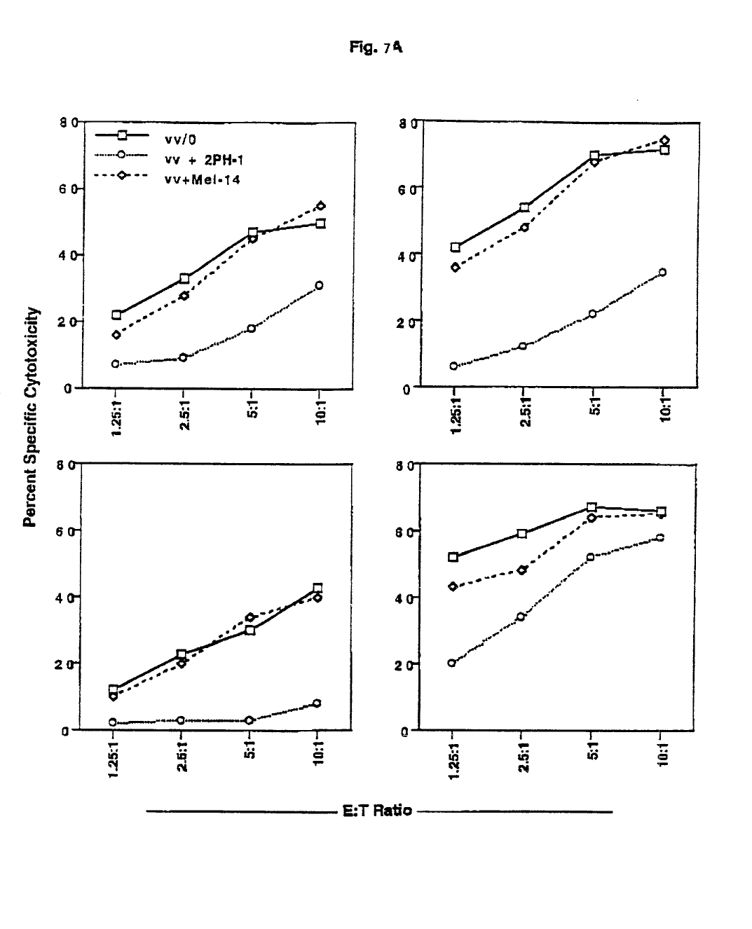

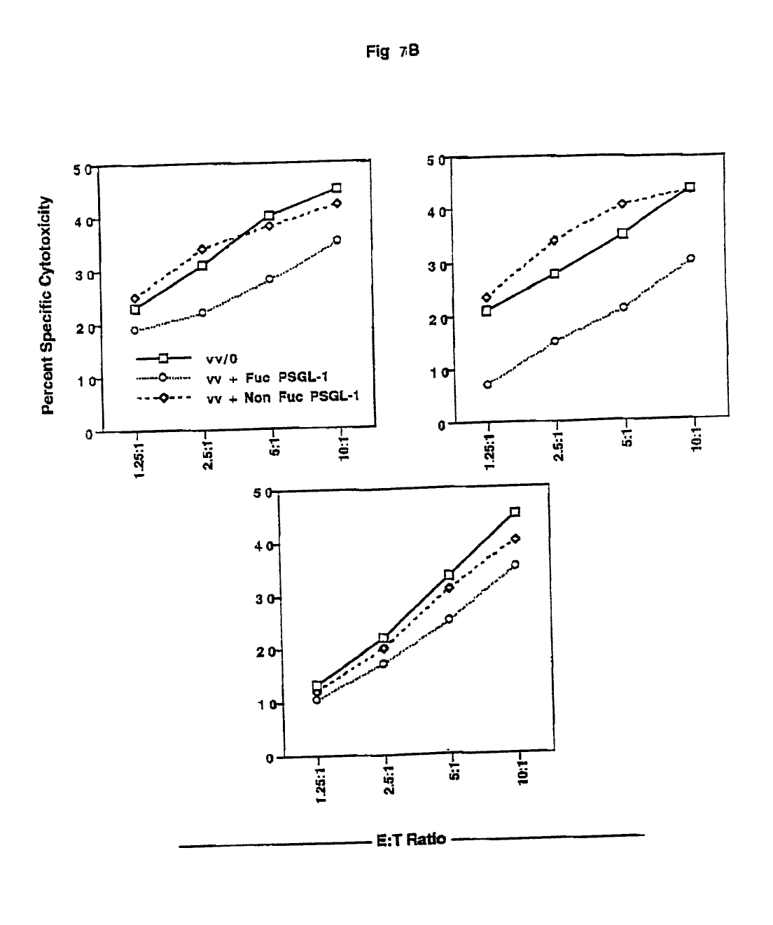

These results strongly suggest that one or more α(1,3)-fucosylated molecule(s) on APC induce(s) the generation of CTL from activated CD8⁺ T cells. One of the candidate molecules we considered was PSGL-1. This sialomucin is expressed on the surface of myeloid and lymphoid cells and can be modified by FTs on many leukocytes including dendritic cells (reviewed in 5). PSGL-1 protein is expressed at normal levels on FT−/−leukocytes, but it is functionally deficient because it lacks the fucosylation needed to serve as a selectin ligand (8 and data not shown). To assess whether fucosylated PSGL-1 was involved in CTL differentiation, we took two approaches. First, we harvested primed splenocytes from vaccinia infected wild-type mice on day 7 p.i. and restimulated the cells with wild-type APC for five days in the presence of mAb 2PH-1 to the N-terminus (aa 42-60) of murine PSGL-1 (30, 31). This mAb significantly inhibited CTL generation, whereas mAb Mel-14 to murine L-selectin (32) had no effect (FIG. 7A). Second, we exposed primed CD8⁺ T cells to vaccinia virus-infected wild-type APC in the presence of a soluble protein consisting of the 40 N-terminal amino acids of human PSGL-1 linked to human Ig heavy chain (PSGL-1/Ig) (33). Recombinant PSGL-1/Ig was either generated in cells that had been cotransfected with core-2 enzyme and FT-VII (to generate PSGL-1/Ig decorated with sLeX-like carbohydrates or from cells that expressed only core-2 enzyme, but not FT-VII (mimicking non-fucosylated PSGL-1 in FT−/−mice). Coincubation with the fucosylated PSGL-1/Ig partially blocked the generation of viral-specific CTL, whereas non-fucosylated PSGL-1/Ig had no effect (FIG. 7B). Importantly, inhibitors of PSGL-1 were only effective when they were present during T cell stimulation by APC. Neither anti-PSGL-1 nor fucosylated PSGL-1/Ig inhibited target cell lysis when they were added only during the CTL assay (not shown).

These findings demonstrate a novel physiological role for the α(1,3)-fucosyltransferases, FT-IV and FT-VII, in APC. Our data suggest that FTs exert this pivotal role by decorating surface-expressed glycoproteins on APC, one of which is PSGL-1. Since anti-PSGL-1 and PSGL-1/Ig were only partially effective in blocking the in vitro generation of CTL from primed wild-type CD8⁺ cells, it cannot be excluded that additional fucosylated molecules exist on APC that may play a similar role. However, mAb 2PH-1 was originally raised against a synthetic peptide resembling the N-terminus of murine PSGL-1 and was selected to block P-selectin/PSGL-1 interactions (30). The finding that CTL activity was normal in selectin−/−mice suggests that activated CD8⁺ cells express counter-receptor(s) for PSGL-1 that must be distinct from the known selectins. It is therefore possible that the hypothetical receptor(s) engage(s) PSGL-1 in a manner that is not entirely inhibitable by mAb 2PH-1. In any event, our results indicate that the manipulation of FTs or PSGL-1 on APC or the putative PSGL-1 receptor(s) on T cells will be useful to control the generation of CD8⁺ effector T cells. This may prove to be a powerful tool to learn more about the generation and function of CTL in vivo. Moreover, our findings may offer a new approach to treat pathologic conditions in humans that are associated with abnormal generation or function of CTL. For example, the ability to selectively modify this critical step might be useful to enhance CTL killer function during viral infections or to combat tumors, whereas CTL suppression might be beneficial for the treatment of autoimmune diseases.

### REFERENCES

1. E. C. Butcher, L. J. Picker. Science 272, 60

2. T. M. Carlos, J. M. Harlan. Blood 84, 2068 (1994).

3. T. A. Springer. Cell 76, 301 (1994).

4. D. Vestweber, J. E. Blanks. Physiol. Rev. 79, 181 (1999).

5. G. S. Kansas. Blood 88, 3259 (1996)

6. J. B. Lowe. Kidney Int. 51, 1418 (1997)

7. P. Maly, A. D. Thall, B. Petryniak, C. E. Rogers, P. L. Smith, R. M. Marks, R. J. Kelly, K. M. Gersten, G. Cheng, T. L. Saunders, S. A. Camper, R. T. Camphausen, F. x. Sullivan, Y. Isogai, O. Hindsgaul, U. H. von Andrian, J. B. Lowe. Cell 86, 643 (1996).

8. John Lowe Unpublised data

9. H. Xie, Y. C. Lim, Luscinskas, A. H. Lichtman. J. Exp. Med. 189, 1765 (1999)

10. S. D. Robinson, P. S. Frenette, H. Rayburn, M. Cummiskey, M. Ullman-Cullere, D. D. wagner, R. O. Hynes. Oroc. Natl. Acad. Sci. USA in press

11. J. R. Bennink, J. W. Yewdell. Curr. Topics Microbiol. Immunol. 163, 153 (1990).

12. M. K. Spriggs, B. H. Koller, T. Sato, P. J. Orrissey, W. C. Fanslow, O. Smithies, R. F. Voice, M. B. Widmer, C. R. Maliszewski. Proc. Natl. Acad. Sci. USA. 89, 6070 (1992).

13. D. Binder, T. M. Kundig. J. Immunol. 146, 4301 (1991).

14. R. V. Blanden. J. Exp. Med. 133, 1091, (1971)

15. D. Kagi, P. Seiler, J. Pavlovic, K. Burki, R. M. Zinkernagel, H. Hengartner. Eur. J. Immunol. 25, 3256 (1995).

16. U. Muller, U. Steinhoff, L. F. L Reis, S. Hemmi, J. Pavlovic, R. M. Zinkernagel, M. Aguet. Science 264, 1918 (1994)

17. D. Kagi, H. Hengartner Curr. Opin. Immunol. 8, 472 (1996)

18. P. S. Frenette, T. N. Mayadas, H. Rayburn, R. O. Hynes, D. D. Wagner. Cell 84, 563 (1996)

19. R. A. Tripp, S. Hou, A. McMickle, J. Houston, P. C. Doherty. J. Immunol. 154, 6013 (1995).

20. D. F. Tough, P. Borrow, J. Sprent. Science 272, 1947 (1996).

21. E. A. Butz, M. J. Bevan. Immunity 8, 167 (1998).

22. K. Murali-Krishna, J. D. Altman, M. Suresh, D. J. D. Sourdive, A. J. Zajec, J. D. Miller, J. Slansky, R. Ahmed. Immunity 8, 177 (1998).

23. G. S. Ogg, X. Jin, S. Bonhoeffer, P. R. Dunbar, M. A. Nowak, S. Monard, J. P. Segal, Y. Cao, S. L. Rowland-Jones, V. Cerundolo, A. Hurley, M. Markowitz, D. D. Ho, D. F. Nixon, A. j. McMichael. Science 279, 2103 (1998).

24. M. L. Arbones, D. C. Old, K. Ley, H. Ratech, C. Maynard-Curry, G. Otten, D. J. Capon, T. F. Tedder. Immunity 1, 247 (1994)

25. Wild-type, FT−/−, and selectin−/−mice (6-8 weeks of age and matched for sex) were infected with the WR strain of vv (ATCC) either sc at the base of the tail or ip (10⁵ pfu/mice in 0.2 ml PBS). On day 7 pi, PEL were harvested by flushing with 3 mls of PBS and /or spleens were collected. Splenocytes and PEL were depleted of RBC by lysis in 0.17 M ammonium chloride and tested for killing of ⁵¹Cr labeled, MC57G targets, uninfected or infected with vv in a standard chromium release assay. Cytotoxicity was defined as (test release-spontaneous release)/(maximum release-spontaneous release)×100%. Percent killing of uninfected targets was subtracted from that of infected targets to calculate viral-specific cytotoxicity.

26. T. N. Mayadas, R. C. Johnson, H. Rayburn, R. O. Hynes, D. D. Wagner. Cell 74, 541, (1993).

27. M. D. Catalina, M. C. Carroll, H. A. Arizpe, A. Takashima, P. Estess, M. H. Siegleman. J. Exp. Med. 184, 2341 (1996).

28. N. Manjunath, P. Shankar, J. Lieberman, U. H. von Andrian. Submitted 29. Mice were infected with vv ip and seven days later, CD8⁺ T cells were positively selected using anti-CD8 antibody-coated Miltenyi beads according to manufacturer's instructions. For APC, splenocytes were depleted of T cells using anti-CD3 coated Miltenyi beads, infected with vv (10 pfu/cell, 2 h at 37° C.), irradiated (400 rads) and UV-treated as described earlier (34). 2×10⁶ CD8⁺ T cells obtained from wild-type or FT−/−mice were cultured with 5×10⁵ wild-type and FT−/−APC in 24-well culture plates for 4-5 days before testing for CTL activity.

30. E. Borges, R. Eytner, R, T. Moll, M. Steegmaier, A. Matthew, I. P. Campbel, K. Ley, H. Mossmann, D. Vestweber. Blood 90, 1934 (1997).

31. Wild type mice were infected with vv ip and 7 days later, splenic CD8⁺ T cells were restimulated with vv-infected APC in 24-well plates as described in ref. 29. At the time of in vitro stimulation, in some cultures soluble recombinant PSGL-1 Ig chimera, its non-fucosylated variant, anti-murine PSGL-1 antibody, 2PH-1, or anti-murine L-selectin antibody, Mel-14 were added at a final concentration of 20 μg/ml. Viral-specific cytotoxicity was determined after 5 days of culture.

32. W. M. Gallatin, I. L. Weissman, E. C Butcher. Nature 304, 30 (1983).

33. M. K. Takada, K. C. Nadeau, G. D. Shaw, K. A. Marquette, Tilney. J. Clin. Invest. 99, 2682 (1997).

34. P. Shankar, J. Fabry, J. Lieberman. Immunol. Invest. 24, 489 (1995).

Table and Figure Legends

Table 1

Total leukocyte counts, T cell subset frequency and activation status of CD8⁺ T cells in PEL, spleen and peripheral blood of wild-type, selectin−/− and FT−/−mice. Mice were infected by i.p. injection of vaccinia virus (10⁵ pfu/mouse) and at day 7 p.i., peripheral blood was obtained by tail bleeding and PEL and spleen were harvested. After lysing of RBC, leukocyte counts were performed on all samples using a hemocytometer. To determine T cell subset proportions, aliquots of cells were labeled with FITC-conjugated anti-CD4 and PE-conjugated anti-CD8 and analyzed on a flow cytometer (FACScan, Becton Dickinson) following standard procedures. To determine the activation status, cells were labeled with anti-CD8 FITC and anti-L-selectin PE or anti-CD8 FITC and anti-CD44 PE. Shown are % CD8⁺ T cells that were L-selectin low or CD44 high. L-selectin levels are not shown for selectin−/−mice because all cells were negative for L-selectin. Mean+/−SD from 6 mice in each group are shown.

FIG. 5

Anti-viral CTL activity and IFN-γ production but not virus-specific proliferation is markedly reduced in FT−/−mice. 5A. CTL activity is reduced in FT−/−but not in selectin−/−mice. Wild-type, triple selectin−/− and FT−/−mice were infected with vv ip and 7 days later, their PEL were tested for lysis of vv infected ⁵¹Cr labeled MC57G target cells (25). Scattergrams for 16 wild-type, 16 FT−/− and 10 selectin−/−mice at 4 different effector: target (E:T) ratios are shown. Each symbol represents the mean percent specific cytotoxicity (from triplicate measurements) of cells from a single animal. 5B. Viral-specific proliferation is comparable in selectin−/− and FT−/−mice. Mice were infected with vv and 7 days later, their splenocytes were immunomagnetically depleted of CD4⁺ T cells and NK cells. 2×10⁵ depleted splenocytes were cultured with equal numbers of T cell-depleted and γ-irradiated splenocytes that were uninfected or infected with vv in triplicate wells of 96-well plates. Two days after stimulation, the cultures were pulsed with ³H thymidine (0.5 μCi/well) for 6-8 h, harvested and counted for ³H incorporation. Shown is the mean cpm+/−S.D. from 3 mice for each strain. 5C. IFN-γ_production is reduced in FT−/−mice but not in selectin−/−mice. PEL obtained on d7 pi were stimulated with 1 μg/ml αCD3 in the presence of Brefeldin A for 6 h, stained with anti-CD8 Cychrome, fixed, permeabilized and then stained with anti-IFN-γ_PE using intracellular staining kit (Pharmingen) before analyzing in a flow cytometer. Representative results from 1 mouse for each strain (out of 3 animals tested with similar results) are shown.

FIG. 6

α(1,3)-fucosylated PSGL-1 is required on APC for the induction of CTL activity in activated CD8⁺ cells.

Wild-type and FT−/−mice were infected with vv and 7 days later, their splenic CD8⁺ T cells were immunomagnetically selected and stimulated with vv-infected wild-type or FT−/−APC (T cell depleted, γ-irradiated splenocytes). Cytotoxicity was measured after 5 days of culture as described in FIG. 5 and ref. 25. Results from 2 mice for each strain are shown.

FIG. 7

Secondary stimulation of CTL activity in primed wild-type CD8⁺ T cells is specifically attenuated in the presence of PSGL-1 blocking antibody or in the presence of recombinant α(1,3)-fucosylated PSGL-1. Wild-type mice were infected with vv and 7 days later, splenocytes were harvested and stimulated with vv in the absence or presence of 20 μg/ml blocking anti-PSGL-1 antibody, 2 PH-1, or control anti-L-selectin antibody, Mel-14 (7A) or in the presence of soluble recombinant fucosylated or non-fucosylated PSGL-1-Ig (7B). Cytotoxicity was determined after 5 days of culture as described in FIG. 5 and ref. 25. Results from four individual mice for 7A and three mice for 7B are shown.

Acknowledgments

This work was supported by National Institute of Health grants HL54936, HL 02881 and HL41484.

TABLE 1

| | Blood × 10⁶/ | | |
|---|---|---|---|
| Total Cells | ml ± S.D. | PEL × 10⁶ ± S.D. | Spleen × 10⁶ ± S.D. |
| +/+ | 5.8 ± 0.8 | 16.1 ± 3.1 | 117.5 ± 11.0 |
| Selectin −/− | 17.5 ± 2.0 | 19.2 ± 3.3 | 262.5 ± 59.0 |
| FT −/− | 20.7 ± 6.3 | 20.75 ± 5.5 | 300 ± 88 |
| CD4⁺ T cells | Percent Total ± S.D. | | |
| +/+ | 12.3 ± 5.5 | 22.9 ± 5.0 | 17.2 ± 2.1 |
| Selectin −/− | 9.24 ± 2.7 | 18.3 ± 4.1 | 17.5 ± 3.5 |
| FT −/− | 10.1 ± 3.2 | 23.8 ± 4.6 | 21.4 ± 4.7 |
| CD8⁺ T cells | Percent Total ± S.D. | | |
| +/+ | 11.1 ± 7.2 | 42.4 ± 14.0 | 10.6 ± 2.6 |
| Selectin −/− | 19.8 ± 7.3 | 50.6 ± 11.5 | 20.9 ± 8.0 |
| FT −/− | 19.3 ± 8.0 | 56.5 ± 6.1 | 21.3 ± 4.6 |

Activation Status of CD8 T cells

| | L- | | L- | | L- | |
|---|---|---|---|---|---|---|
| | selectin | CD44 | selectin | CD44 | selectin | CD44 |
| | low | high | low | high | low | high |
| +/+ | 73 ± 8 | 86 ± 5 | 85 ± 7 | 96 ± 1 | 58 ± 5 | 59 ± 3 |
| Selectin −/− | | 89 ± 6 | | 98 ± 2 | | 62 ± 3 |
| FT −/− | 79 ± 5 | 92 ± 3 | 83 ± 3 | 98 ± 1 | 54 ± 5 | 61 ± 7 |

1. A method of inhibiting the differentiation of an activated T-cell into a cytotoxic lymphocyte in a mammalian subject, said method comprising administering to said subject a therapeutically effective amount of a PSGL antagonist.

2. The method of claim 1, wherein said PSGL antagonist is selected from the group consisting of a soluble form of PSGL, an antibody directed to PSGL, an antibody directed to sLeₓ, an antibody directed to sulfated tyrosine, sLeₓ, mimetics which inhibit sLeₓ binding and a small molecule inhibitor of PSGL binding.

3. The method of claim 2, wherein said PSGL antagonist is a soluble form of PSGL.

4. The method of claim 2, wherein said PSGL antagonist is an antibody directed to PSGL.

5. A method of treating or ameliorating an autoimmune condition, said method comprising administering to said subject a therapeutically effective amount of a PSGL antagonist.

6. The method of claim 5, wherein said PSGL antagonist is selected from the group consisting of a soluble form of PSGL, an antibody directed to PSGL, an antibody directed to sLeₓ, an antibody directed to sulfated tyrosine, sLeₓ, mimetics which inhibit sLeₓ binding and a small molecule inhibitor of PSGL binding.

7. The method of claim 6, wherein said PSGL antagonist is a soluble form of PSGL.

8. The method of claim 6, wherein said PSGL antagonist is an antibody directed to PSGL.

9. A method of treating or ameliorating a allergic reaction, said method comprising administering to said subject a therapeutically effective amount of a PSGL antagonist.

10. The method of claim 9, wherein said PSGL antagonist is selected from the group consisting of a soluble form of PSGL, an antibody directed to PSGL, an antibody directed to sLeₓ, an antibody directed to sulfated tyrosine, sLeₓ, mimetics which inhibit sLeₓ binding and a small molecule inhibitor of PSGL binding.

11. The method of claim 10, wherein said PSGL antagonist is a soluble form of PSGL.

12. The method of claim 10, wherein said PSGL antagonist is an antibody directed to PSGL.

13. A method of treating or ameliorating asthma, said method comprising administering to said subject a therapeutically effective amount of a PSGL antagonist.

14. The method of claim 13, wherein said PSGL antagonist is selected from the group consisting of a soluble form of PSGL, an antibody directed to PSGL, an antibody directed to sLeₓ, an antibody directed to sulfated tyrosine, sLeₓ, mimetics which inhibit sLeₓ binding and a small molecule inhibitor of PSGL binding.

15. The method of claim 14, wherein said PSGL antagonist is a soluble form of PSGL.

16. The method of claim 14, wherein said PSGL antagonist is an antibody directed to PSGL.

17. The method of claim 3, wherein said soluble form of PSGL comprises the first 19 amino acids of the mature amino acid sequence of PSGL.

18. The method of claim 17, wherein said soluble form of PSGL comprises the first 47 amino acids of the mature amino acid sequence of PSGL.

19. The method of claim 18, wherein said 47 amino acids are fused to the Ig portion of an immunoglobulin chain.

20. The method of claim 7, wherein said soluble form of PSGL comprises the first 19 amino acids of the mature amino acid sequence of PSGL.

21. The method of claim 20, wherein said soluble form of PSGL comprises the first 47 amino acids of the mature amino acid sequence of PSGL.

22. The method of claim 21, wherein said 47 amino acids are fused to the Ig portion of an immunoglobulin chain.

23. The method of claim 11, wherein said soluble form of PSGL comprises the first 19 amino acids of the mature amino acid sequence of PSGL.

24. The method of claim 23, wherein said soluble form of PSGL comprises the first 47 amino acids of the mature amino acid sequence of PSGL.

25. The method of claim 24, wherein said 47 amino acids are fused to the Ig portion of an immunoglobulin chain.
